(19) United States
(12) Patent Application Publication
(10) Pub. No.: US 2008/0004630 A1
(43) Pub. Date: Jan. 3, 2008

(54) Multiple-tract cannulas and related methods thereof

(75) Inventor: Behnam Badie, LaCanada, CA (US)

Correspondence Address: Medlen & Carroll, LLP;Suite 350, 101 Howard Street, San Francisco, CA, 94105, US

(73) Assignee: Madison Surgical Designs, LLC, La Canada, CA (US)

(21) Appl. No.: 11/651,775

(22) Filed: Jan. 10, 2007

Related U.S. Application Data
(60) Provisional application No. 60/757,685, filed on Jan. 10, 2006.

(51) Int. Cl. A61M 25/00

(52) U.S. Cl. 606/108

(57) ABSTRACT

The present invention relates to multiple-tract cannula devices and related methods of use. In certain embodiments, the present invention relates to multiple-tract cannula devices used for contacting and treating brain tissue (e.g., delivering pharmaceutical agents to brain tissue).

The present application claims priority to U.S. Provisional Application 60/757,685 filed Jan. 10, 2006, which is herein incorporated by reference.

### FIELD OF THE INVENTION

The present invention relates to multiple-tract cannula devices and related methods of use. In certain embodiments, the present invention relates to multiple-tract cannula devices used for contacting and treating brain tissue (e.g., delivering pharmaceutical agents to brain tissue).

### BACKGROUND

The presence of the blood-brain barrier (BBB) prevents the penetration of large molecules into brain tumors. This limitation is most pronounced with high molecular weight and polar molecules. Many new drugs with therapeutic potential include polar proteins with a high molecular weight (e.g., growth factors, enzymes, antibodies, protein conjugates, genetic vectors). One strategy to circumvent the BBB and to improve drug delivery to the CNS is direct administration into the CSF or the brain parenchyma. However, ventriculocisternal perfusion relies on diffusion for distribution into the brain parenchyma and poor penetration occurs, even with drugs with ideal characteristics for diffusion. Penetration from CSF into the brain is extremely limited for high molecular weight compounds. After direct administration into the brain, limited diffusion in the brain severally retards the rate of distribution of such compounds.

Intratumoral chemotherapy for brain tumors has been administered by direct injection, intracavitary instillation, intracavitary topical application, chronic low-flow microinfusion, and controlled release from polymer implants. Intracavitary delivery has also been used to deliver biological reagents, including interferons and interleukin-2, to tumor. However, the efficacy of intratumoral drug administration is restricted by the poor diffusion of drug through tumor and brain interstitium relative to tissue clearance so that only a small volume of tissue surrounding the drug source is treated. Thus, poor drug delivery to the CNS limits application of promising basic findings for further research and treatment of neurological diseases such as neurodegenerative disorders, central nervous system tumors and inborn errors of metabolism.

Neurosurgery, however, demands special considerations. Obtaining surgical access to brain tumors for purposes of drug delivery generally requires the creation of multiple openings in the skull (called a craniotomy). Most often, a craniotomy involves a large incision and dissection of other soft tissue that results in significant postoperative pain and discomfort. Furthermore, reaching tumors in the brain requires openings into the surface of the brain itself. This brain dissection and manipulation can result in neurological deficits.

What is needed are improved methods of delivering compounds, such as high molecular weight compounds, to locations of the brain. In addition, what is needed are improved devices capable of providing multiple points of access to a particular brain region for purposes of drug delivery through a single brain opening.

### SUMMARY OF THE INVENTION

The present invention relates to multiple-tract cannula devices and related methods of use. In certain embodiments, the present invention relates to multiple-tract cannula devices used for contacting and treating brain tissue (e.g., delivering pharmaceutical agents to brain tissue).

In some embodiments, the present invention provides a cannula device comprising a tubular extension and an attachment member, wherein the tubular extension comprises proximal and distal ends and an exterior surface, the tubular extension having a longitudinal axis and having therein at least two hollow tracts running from the proximal end and through the exterior surface, and wherein the attachment member is connected to the proximal end of the tubular extension, and wherein the attachment member comprises an overhang portion that extends beyond the exterior surface of the tubular extension. In certain embodiments, the present invention provides a cannula device comprising a tubular extension and an attachment member, wherein the tubular extension comprises proximal and distal ends and an exterior surface, the tubular extension having a longitudinal axis and having therein at least two hollow tracts running from the proximal end and through the exterior surface, wherein the tubular extension is inflexible, and wherein the attachment member is connected to the proximal end of the tubular extension, and wherein the attachment member comprises an overhang portion that extends beyond the exterior surface of the tubular extension.

In certain embodiments, the present invention provides a cannula device comprising a tubular extension, wherein the tubular extension comprises proximal and distal ends and an exterior surface, the tubular extension having a longitudinal axis and having therein at least two hollow tracts running in separate spiral trajectory paths from the proximal end and through the exterior surface. In certain embodiments, at least two of the separate spiral trajectory paths are parallel. In particular embodiments, the present invention provides a cannula device comprising a tubular extension and an attachment member, wherein the tubular extension comprises proximal and distal ends and an exterior surface, the tubular extension having a longitudinal axis and having therein at least two hollow tracts running from the proximal end and through the exterior surface, wherein the hollow tracks extend through the tubular extension in a non-parallel manner with respect to one another, and wherein the attachment member is connected to the proximal end of the tubular extension, wherein the attachment member comprises an overhang portion that extends beyond the exterior surface of the tubular extension.

In certain embodiments, the present invention provides a method of delivering pharmaceutical agents, or other relevant agents, to a tissue protected by the blood-brain barrier (e.g. brain tissue mass), comprising providing a subject with a target tissue mass; a cannula device secured within a burr hole of the subject's cranium such that the cannula device is contacting the target tissue, the cannula device comprising an tubular extension having therein at least two hollow tracts running through the tubular extension; and at least two surgical catheters; and guiding the surgical catheters through the hollow tracts of the tubular extension such that the surgical catheters are positioned in the vicinity of the target tissue; and administering the agent or agents through the surgical catheters onto the target tissue. In certain embodiments, the internal body cavity has therein a tumor, an infected region, or a region requiring administration of pharmaceutical agents.

In certain embodiments, the present invention provides a kit comprising a cannula device comprising a tubular extension and an attachment member, wherein the tubular extension comprises proximal and distal ends and an exterior surface, the tubular extension having a longitudinal axis and having therein at least two hollow tracts running from the proximal end and through the exterior surface, wherein the tubular extension is inflexible, is less than 7.5 cm in length, or is inflexible and less than 7.5 cm in length, and wherein the attachment member is connected to the proximal end of the tubular extension, wherein the attachment member comprises an overhang portion that extends beyond the exterior surface of the tubular extension, and wherein the overhang portion has therein a plurality of holes configured to receive fastening agents (although in some embodiments the overhang portion is configured for attachment to a bone with an adhesive agent (e.g., glue)); and a surgical catheter or the fastening agents.

In certain embodiments, the present invention provides a kit comprising a cannula device comprising a tubular extension, wherein the tubular extension comprises proximal and distal ends and an exterior surface, the tubular extension having a longitudinal axis and having therein at least two hollow tracts running in separate spiral trajectory paths from the proximal end and through the exterior surface; and a surgical catheter. In certain embodiments, at least two of the separate spiral trajectory paths are parallel. In certain embodiments, at least two of the separate spiral trajectory paths are non-parallel.

In certain embodiments, the present invention provides a method of delivering pharmaceutical agents to a body cavity, comprising providing a subject with a body cavity; a cannula device secured within a bone hole of the subject such that the cannula device is contacting the body cavity, the cannula device comprising an tubular extension having therein at least two hollow tracts running through the tubular extension; and at least two surgical catheters; and guiding the surgical catheters through the hollow tracts of the tubular extension such that the surgical catheters are positioned in the vicinity of the body cavity; and administering the pharmaceutical agents through the surgical catheters onto the body cavity.

In certain embodiments, the present invention provides a cannula device comprising a tubular extension and an attachment member, wherein the tubular extension comprises proximal and distal ends and an exterior surface, the tubular extension having a longitudinal axis and having therein at least two hollow tracts running from the proximal end and through the exterior surface, wherein the tubular extension is inflexible, and wherein the attachment member is connected to the proximal end of the tubular extension, wherein the attachment member comprises an overhang portion that extends beyond the exterior surface of the tubular extension. In certain embodiments, the present invention provides methods of performing surgical operations using the above cannula, wherein the attachment member is glued, or otherwise attached, to the surface of a bone after the cannula is inserted through a hole in the bone.

In certain embodiments, the overhang portion has therein a plurality of holes configured to receive fastening agents. In certain embodiments, the overhang portion is configured for attachment to a bone with, for example, an adhesive agent (e.g., glue). In certain embodiments, wherein the diameter of the hollow tracts is less than 9 mm or less than 5 mm (e.g. 8, 7, 6, 5, 4, 3, 2 or 1 mm). In certain embodiments, the diameter of the hollow tracts is between 1 and 3 mm. In certain embodiments, the diameter of the tubular extension is less than 5 cm in diameter (e.g. 4.5, 4, 3.5, 3, 2.5, 2, 1.5, or 1 cm). In certain embodiments, the diameter of the tubular extension is between 1 and 2 cm. In certain embodiments, the tubular extension has two of the hollow tracts. In certain embodiments, the tubular extension has three, four, five, six, seven, or more of the hollow tracts.

In certain embodiments, the overhang portion of the attachment member comprises a first overhang flap on one side of the attachment member and a second overhang flap across from the first overhang flap, wherein each of the first and second overhang flaps contains at least one hole therein configured for receiving a fastening agent. In certain embodiments, the overhang portion of the attachment member extends beyond the exterior surface of the tubular extension throughout the full circumference (or nearly the full circumference) of the exterior surface of the tubular extension.

In certain embodiments, the number of the hollow tracts within the tubular extension is selected from the group consisting of 2, 3, 4, 5, 6, 7, 8, 9, 10, or more (e.g. 20, 30, 40, 50, or more, such as 1000 if micro-tracts are employed). In certain embodiments, at least two of the hollow tracts extend through the tubular extension in a non-parallel manner with respect to one another. In certain embodiments, at least two of the hollow tracts extend through the tubular extension in separate spiral trajectory paths. In certain embodiments, at least two of the separate spiral trajectory paths are parallel. In certain embodiments, at least two of the separate spiral trajectory paths are non-parallel. In certain embodiments, the tubular extension is less than 7.5 centimeters in length (e.g., 7.0 cm in length, 5.0 cm in length, 3.0 cm in length, 2.0 cm in length or 1.0 cm in length). In certain embodiments, the tubular extension is between 1 and 5 cm in length (e.g. 2-3 cm in length).

In certain embodiments, the attachment member is rigidly connected to the proximal end of the tubular extension. In certain embodiments, the attachment member is connected at an angle between 0 and 180 degrees in relation to the longitudinal axis. In certain embodiments, the attachment member is flexibly attached to the proximal end of the tubular extension. In certain embodiments, the attachment member may be adjusted to an angle between 0 and 180 degrees in relation to the longitudinal axis. In certain embodiments, the cannula device further comprises a hinge, wherein the attachment member attaches to the proximal end of the tubular extension with the hinge. In certain embodiments, the hollow tracts are configured to receive surgical catheters. In certain embodiments, the cannula device has therein a removable stylet.

### BRIEF DESCRIPTION OF THE DRAWINGS

FIG. 1 shows a side view of a multiple-tract cannula device embodiment of the present invention.

FIG. 2 shows a cross section view of a multiple tract cannula device. FIGS. 3A shows a cross section view of a multiple-tract cannula device, and FIG. 3B schematically displays tubular extension hollow tracts with spiral trajectory paths.

FIGS. 4A, 4B, 4C and 4D show a top-down perspective of an attachment member.

FIGS. 5A and 5B show a bottom-up perspective of a cannula device.

FIG. 6 shows a side perspective of a cannula device

FIG. 7 shows a bottom-up perspective of a cannula device.

FIG. 8 shows a cannula device positioned in the proximity of a brain mass.

FIG. 9 shows a cannula device positioned in the proximity of a brain mass.

### DEFINITIONS

To facilitate an understanding of the invention, a number of terms are defined below.

As used herein, the term “bone hole” refers to a surgically inserted hole through a bone (e.g., cranium, mandible, cervical vertebrae, clavicle, scapula, sternum, ribs, humerus, thoracic vertebrae, lumbar vertebrae, ulna, radius, pelvis, carpals, phalanges, sacrum, metacarpals, femur, patella, tibia, fibula, tarsals, metatarsals).

As used herein, the term “burr hole” refers to a surgically inserted hole through the cranium. Generally, a “burr hole” is utilized during neurosurgical procedures.

As used herein, the term “subject” refers to any animal. Examples of subject include, but are not limited to, cats, dogs, mice, primates, humans, birds, and fish.

As used herein, the term “tissue” refers to a part of a group of cells within a subject's body. An example of a tissue is a brain tumor.

As used herein, the term “tumor” refers to an abnormal mass of tissue that results from, for example, excessive cell division.

As used herein, the term “brain tumor” refers to a tumor located within a subject's brain. Examples of brain tumors include, but are not limited to, astrocytoma tumors, glioma tumors, atypical teratoid/rhabdoid tumors, brain stem gliomas, choroid plexus tumors, craniopharyngiomas, ependymoma tumors, ganglioglioma tumors, germ cell tumors, gliomatosis cerbri tumors, infant brain tumors, medulloblastoma tumors, oligodendroglioma tumors, and optic pathway tumors. Brain tumors can be located near a subject's cranium surface (e.g., within 2 cm of the subject's cranium surface) or located in a deeply seated region of the brain (e.g., at least 2 cm away from the subject's cranium surface).

As used herein, the term “deeply seated brain tumor” refers to brain tumors located in difficult to reach areas of the brain (e.g., located at least 2 cm away from the subject's cranium surface). Examples of deeply seated brain tumors include, but are not limited to, brain tumors located in the brain stem, brain tumors located in the thalamus, brain tumors located in the motor area, and brain tumors located in the deep areas of gray matter.

As used herein, the term “resection” refers to excision of a portion or all of a tissue structure.

As used herein, the term “biopsy” refers to a procedure that involves obtaining a tissue specimen to establish a precise diagnosis. Biopsies can be accomplished, for example, with a biopsy needle or by an open surgical incision.

As used herein, the term “brain cancer” refers to forms to cancer located within a subject's brain. Examples of brain cancer include, but are not limited to, childhood brain stem glioma, childhood cerebellar astrocytoma, childhood cerebral astrocytoma/malignant glioma, childhood ependymoma, childhood medulloblastoma, childhood supratentorial primitive neuroectodermal tumors and pineoblastoma, and visual pathway and hypothalamic glioma.

As used herein, the term “inflexible” in reference to a tubule extension, refers to a tubule extension that is rigid such that it is resistant to bending about its longitudinal axis. A tubule extension is considered inflexible if it cannot be bent about its longitudinal axis unless such force is applied that causes a permanent bend in the tubule extension.

### DETAILED DESCRIPTION

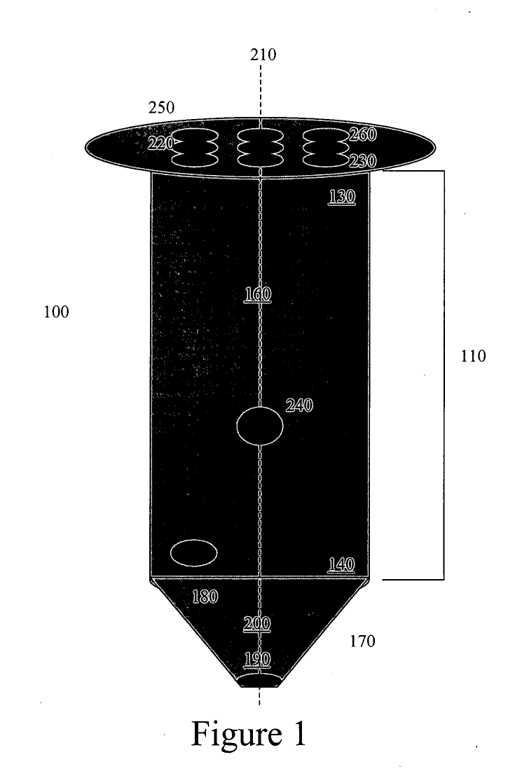

The present invention relates to cannula devices, kits, and related methods of use. In particular, the present invention relates to cannula devices used for surgical procedures, such as contacting and treating brain tissue (e.g., delivering pharmaceutical agents to brain tissue), treating liver diseases, as well as other types of organs and tissues. FIGS. 1-9 illustrate various preferred embodiments of the cannula devices and related methods thereof. The present invention is not limited to these particular embodiments.

The illustrated embodiments describe the cannula devices of the present invention in terms of neurosurgical applications (e.g., delivering pharmaceutical agents to brain tissue). However, it should be appreciated that the cannula devices are not limited to neurosurgical applications. Indeed, the cannula devices of the present invention have application in any procedure requiring access to a body cavity (e.g., spinal surgery, bone marrow applications, liver tumor surgery, etc.). The devices of the present invention provide advantages over the prior art.

Generally, delivery of pharmaceutical agents (e.g., drugs) to a portion of the brain (e.g., a brain tumor) involves the slow infusion of the pharmaceutical agents through multiple catheters through separate burr holes. As such, delivery of pharmaceutical agents to a variety of different positions within a brain mass requires the use of multiple catheters positioned within multiple burr holes. The cannula devices of the present invention overcome this limitation within the prior art. In particular, the cannula devices of the present invention allow the positioning of multiple drug delivery catheters directed at a variety of angles in relation to a brain mass (or other bodily tissue). As such, the cannula devices of the present invention optimize drug delivery to the brain while minimizing the risk associated with multiple catheters positioned within multiple burr holes.

FIG. 1 shows a side view of a cannula device 100 embodiment of the present invention. The cannula device 100 is not limited to a particular material composition (e.g., synthetic rubber, titanium, biocompatible plastic, polyacetal, elastomeric material, polyurethane, polyethylene, stainless steel, metal, or mixture thereof). In certain embodiments, the material composition of the cannula device 100 comprises polyacetal or other rigid material. The cannula device 100 is not limited to particular size dimensions. In certain embodiments, the size of the cannula device 100 is dependent upon its desired use (e.g., neurosurgical applications, liver tumor surgery applications, etc.). For example, cannula devices 100 (e.g., designed for neurosurgical procedures) may range in size from, for example, 0.1 to 10 cm in length and 0.1 to 5 cm in diameter. In certain embodiments, the size dimensions for cannula devices 100 (e.g., designed for neurosurgical applications) ranges from 1 to 5 cm in length and 1 to 2 cm in diameter. Multiple-tract cannula devises 100 designed for general surgery applications (e.g., liver surgery) range in size from, for example, 1 to 40 cm in length and 0.1 cm to 10 cm in diameter. In certain embodiments, the size of cannula devices 100 designed for general surgical applications (e.g., liver surgery) ranges from 5 to 20 cm in length and 1 to 3 cm in diameter. In certain embodiments, the cannula devices 100 are configured to engage and attach within a bone hole (e.g., a burr hole). In certain embodiments, the cannula devices 100 are configured to provide multiple access points to an interior body cavity (e.g., a brain tumor or other type of tissue).

Still referring to FIG. 1, in some embodiments, the cannula devices 100 generally comprise a tubular extension 110. The tubular extension 110 has a tubular extension body proximal end 130, a tubular extension body distal end 140, and a tubular extension body exterior surface 160; a tubular extension nose 170 having a tubular extension nose proximal end 180, a tubular extension nose distal end 190, and a tubular extension nose exterior surface 200; and a tubular extension longitudinal axis 210. In certain embodiments, unlike surgical catheters that are flexible, the tubular extension 110 is inflexible. The tubular extension 110 is not limited to particular size dimensions. In certain embodiments, the size of the tubular extension 110 is dependent upon the desired use of the cannula device 100 (e.g., neurosurgical applications, liver tumor surgery applications, etc.). For example, a cannula device 100 designed for neurosurgical procedures may have, for example, a tubular extension 110 ranging in size from 0.1 to 10 cm in length and 0.1 to 5 cm in diameter. In certain embodiments, a cannula device 100 designed for neurosurgical applications may have a tubular extension 110 ranging in size from 1 to 5 cm in length and 1 to 2 cm in diameter. A cannula device 100 designed for general surgical procedures (e.g., liver surgery) may have a tubular extension 110 ranging in size from, for example, 1 to 40 cm in length and 0.1 cm to 10 cm in diameter. In certain embodiments, a cannula device 100 designed for general surgical applications (e.g., liver surgery) may have a tubular extension 110 ranging in size from 5 to 20 cm in length and 1 to 3 cm in diameter. In certain embodiments, the length of the tubular extension 110 may be varied so that it can reach lesions (e.g., tumors, vascular malformations, infections, blood clots, etc.) of different depths. The diameter of the tubular extension 110 is preferably kept as small as possible to minimize, for example, the amount of bone that is removed and to minimize exposure of the body cavity to the environment.

Still referring to FIG. 1, in certain embodiments, as a cannula device 100 is positioned within a bone hole (e.g., burr hole), the tubular extension 110 fits within the bone hole such that the tubular extension exterior body exterior surface 160 engages both the interior sides of the bone hole and the interior body cavity situated beyond the bone hole (e.g., the brain). In certain embodiments, the tubular extension 110 may be positioned at any angle with respect to the bone hole (e.g., 90, 80, 70, 60, 50, 40, 30, 20, 10, 1 degree angle).

Still referring to FIG. 1, the tubular extension 110 is not limited to a particular shape (e.g., spherical, oval, conical). In certain embodiments, the shape of the tubular extension 110 is cylindrical. In certain embodiments, the tubular extension 110 has a navigation system (see, e.g., Medtronic Neurosurgery navigation products including, but not limited to, StealthStation TREON plus Medtronic Navigational System, StealthStation TRIA plus Medtronic Navigational System, and StealthStation AXIEM Electromagnetic Medtronic Navigational System) positioned on the tubular extension body proximal end 130 facilitating accurate insertion of the cannula device 100 into a body cavity.

Still referring to FIG. 1, the tubular extension nose 170 is positioned such that the tubular extension nose proximal end 180 engages the tubular body extension distal end 140. In certain embodiments, the size of the tubular extension nose proximal end 180 matches the size of the tubular extension distal end 140. The tubular extension nose 170 is not limited to a particular shape. In certain embodiments, the tubular extension nose 170 is funnel shaped such that the size of the tubular extension nose 170 gradually diminishes from the tubular extension nose proximal end 180 to the tubular extension nose distal end 190. In some embodiments, the tubular extension nose 170 has an endoscopic camera positioned on the tubular extension nose distal end 190 (see, e.g., Pediatric Endoscope System, Richard Wolf Neuroendoscopy; herein incorporated by reference in its entirety). In some embodiments, the tubular extension nose 170 has therein a removable stylet. The tubular extension nose 170 is not limited to a particular type, size, or shape of stylet. In certain embodiments, the stylet assists in navigating the cannula device 100 through the brain or other region of the body. In certain embodiments, the stylet is a rigid metal shaft with drill or screw type structures and a robust (e.g., hardened) end (e.g., pointed end, curved end). Other suitable stylet configurations are described in, for example, U.S. Pat. No. 6,033,411 and U.S. Patent Application Publication No. 2002-0188300, each of which is herein incorporated by reference in their entireties. In certain embodiments, the stylet is removed after positioning of the cannula device 100 within a body cavity (e.g., the brain).

Still referring to FIG. 1, the tubular extension 110 has therein multiple (e.g., 2, . . . 5, . . . 9, 10, or more) tubular extension hollow tracts 220 running through the tubular extension 110 and tubular extension nose 170. In certain embodiments, each tubular extension hollow tract 220 has a tubular extension hollow tract entrance 230 and a tubular extension hollow tract exit 240. The tubular extension 110 is not limited to a particular position location for each tubular extension hollow tract entrance 230 and tubular extension hollow tract exit 240. In certain embodiments, the tubular extension hollow tract exits 240 are positioned along the tubular extension body exterior surface 160 and/or the tubular extension nose exterior surface 200. The tubular extension hollow tracts 220 are not limited to a particular diameter (e.g., 0.1 to 10 mm, or larger). In certain embodiments, the diameter of the tubular extension hollow tracts 220 is approximately 1 to 3 mm. In certain embodiments, the diameter of the tubular extension hollow tracts 220 is such that it can receive and guide surgical catheters.

Still referring to FIG. 1, the tubular extension hollow tracts 220 are not limited to particular trajectory paths. In certain embodiments, the tubular extension hollow tracts 220 assume trajectory paths that are parallel with the tubular extension longitudinal axis 210. In certain embodiments, the tubular extension hollow tracts 220 assume trajectory paths that begin in parallel with the tubular extension longitudinal axis 210 but angle away (e.g., between 0.01 to 179.99 degrees) from the tubular extension longitudinal axis 210 at the tubular extension hollow tract exit 240. In certain embodiments, the tubular extension hollow tracts 220 assume trajectory paths at an angle (e.g., between 0.01 to 179.99 degrees) from the tubular extension longitudinal axis 210. In certain embodiments, the tubular extension hollow tracts 220 assume spiraling (e.g., corkscrewing) trajectory paths in relation in to the tubular extension longitudinal axis 210. In certain embodiments, the tubular extension hollow tracts 220 are interconnected within a tubular extension 110. In certain embodiments, the tubular extension hollow tracts 220 are not interconnected. In certain embodiments, some tubular extension hollow tracts 220 within a tubular extension 110 are interconnected, while other tubular extension hollow tracts 220 within a tubular extension 110 are not interconnected. In certain embodiments, the tubular extension hollow tracts 220 facilitate the positioning of surgical catheters in the vicinity of body cavities (e.g., brain tumors) for medical purposes (e.g., drug delivery to the body cavity). In some embodiments, at least two of the hollow tracts 220 extend through the tubular extension 110 in a non-parallel manner with respect to one another.

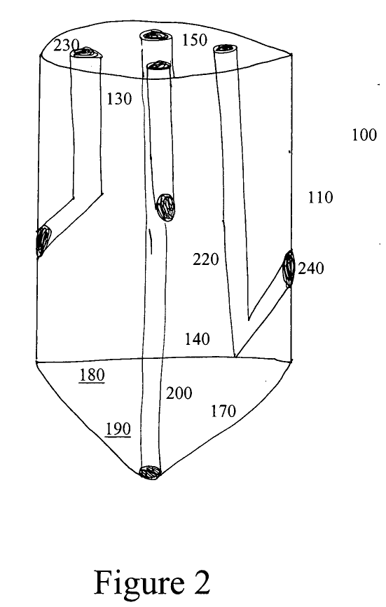

FIG. 2 shows a cross section view of a tubular extension 110 having a tubular extension body proximal end 130, a tubular extension body distal end 140, a tubular extension body proximal face 150; a tubular extension nose 170 having a tubular extension nose proximal end 180, a tubular extension nose distal end 190, and a tubular extension nose exterior surface 200; a tubular extension longitudinal axis 210; and multiple tubular extension hollow tracts 220 having tubular extension hollow tract entrances 230 and tubular extension hollow tract exits 240. As shown in FIG. 2, the tubular extension hollow tracts 220 need not assume uniform trajectory paths. For example, some tubular extension hollow tracts 220 assume a trajectory path in parallel with the tubular extension longitudinal axis, while other tubular extension hollow tracts 220 assume trajectory paths beginning in parallel with the tubular extension longitudinal axis but angling away (e.g., between 0.01 to 179.99 degrees) from the tubular extension longitudinal axisat the tubular extension hollow tract exit 240. In certain embodiments, the variety of trajectory paths for the tubular extension hollow tracts 220 permits the positioning of surgical catheters at varied positions surrounding a body cavity (e.g., a brain tumor).

FIGS. 3A and 3B show a cross section view of a tubular extension 110 having a tubular extension body proximal end 130, a tubular extension body distal end 140, a tubular extension body proximal face 150; a tubular extension nose 170 having a tubular extension nose proximal end 180, a tubular extension nose distal end 190, and a tubular extension nose exterior surface 200; a tubular extension longitudinal axis 210; and multiple tubular extension hollow tracts 220 having tubular extension hollow tract entrances 230 and tubular extension hollow tract exits 240. As shown in FIG. 3A, the tubular extension hollow tracts 220 assume spiraling (e.g., corkscrewing) trajectory paths in relation in to the tubular extension longitudinal axis 210. In certain embodiments, a series of tubular extension hollow tracts 220 positioned in a spiraling manner permits the positioning of surgical catheters at varied positions surrounding a body cavity (e.g., a brain tumor). FIG. 3B schematically displays a series of tubular extension hollow tracts 220 following spiral trajectory paths. As shown in FIG. 3B, each tubular extension hollow tract following a spiral trajectory path has a separate tubular extension hollow tract exit 240. In addition, as shown in FIG. 3B, the tubular extension hollow tracts following spiral trajectory paths are positioned in a parallel manner.

Referring again to FIG. 1, in certain embodiments, the cannula device 100 comprises an attachment member 250 connected to the tubular extension body proximal end 130. The attachment member may be used to secure the cannula device 100 to the surface area of a subject (e.g. attaching the cannula device 100 to a bone). The attachment member 250 is not limited to a particular position in relation to the tubular extension body proximal end 130. In certain embodiments, the attachment member 250 extends outward from the tubular extension body proximal end 130 at any desired angle (e.g., 0 to 180 degrees). The attachment member 250 is not limited to a particular shape (e.g., circular, oval, triangular, square, diagonal, rectangular, etc.). In certain embodiments, the shape of the attachment member 250 is a circular protruding lip. The attachment member 250 is not limited to a particular extension length. In certain embodiments, the extension length of the attachment member 250 is at least 0.1 cm (e.g., at least 0.5 cm, 1 cm . . . 2 cm). In some embodiments, the attachment member 250 has protruding lip fixtures (e.g., edges, lips, tongues, etc.) allowing additional medical instruments (e.g., surgical catheters) to lock onto the cannula device 100 and prevent undesired movement of the medical instrument.

In some embodiments, the attachment member 250 has protruding lip fixtures (e.g., edges, lips, tongues, etc.) allowing frameless stereotactic navigation systems such as StealthStation (Medtronic Navigation, Colorado) or BrainLAB VectorVision (Germany) to lock onto the cannula device 100. Attachment of the navigation system will allow for planning of catheter placement and predict catheter positioning before catheters are passed through the cannula.

Still referring to FIG. 1, the attachment member 250 has therein attachment member openings 260. The attachment member 250 is not limited to a particular number of attachment member openings 260. In certain embodiments, the number of attachment member openings 260 is consistent with the number of tubular extension hollow tracts 220. The attachment member openings 260 are not limited to a particular shape. In certain embodiments, the shape of the attachment member openings 260 is consistent with the shape of the tubular extension hollow tracts 220. The attachment member openings 260 are not limited to a particular size. In certain embodiments, the size of the attachment member openings 260 are consistent with the size of the tubular extension hollow tracts 220. The attachment member openings 260 are not limited to a particular positioning on the attachment member 250. In certain embodiments, the positioning of the attachment member openings 260 on the attachment member 250 is consistent with the positioning of the tubular extension hollow tract entrances 230 on the tubular extension body proximal face 150.

Still referring to FIG. 1, in certain embodiments, as a cannula device 100 is positioned within a bone hole (e.g., burr hole), the attachment member 250 engages and attaches onto the top surface of the bone thereby securing the cannula device 100 within the bone. In some embodiments, the attachment member 250 attaches onto the top surface of a bone with an adhesive agent (e.g., fibrin glue, cranioplastic cement). In certain embodiments, the attachment member 250 attaches onto the top surface of a bone with fastening agents (e.g., nails, threaded fasteners, screws etc.).

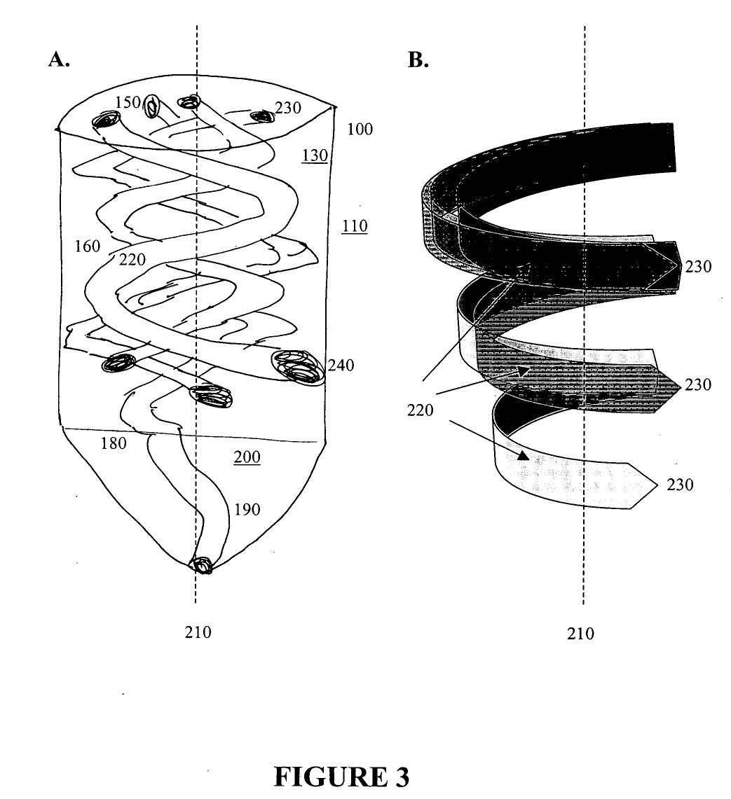

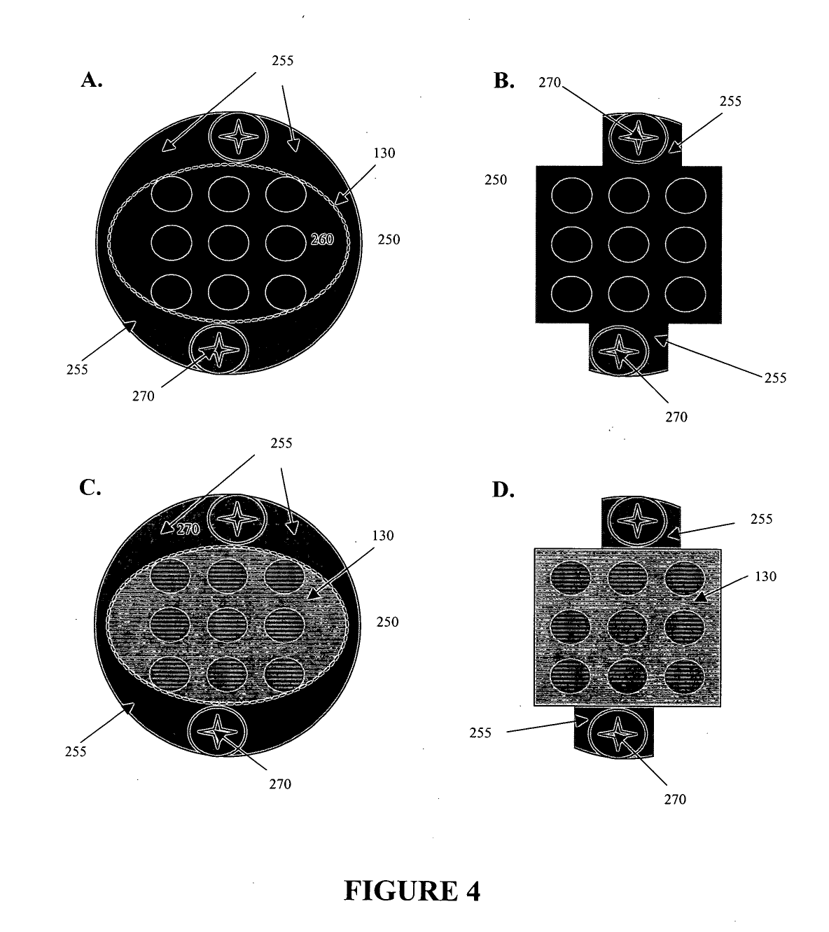

FIG. 4A shows a top-down perspective of an attachment member 250 having attachment member openings 260 and attachment holes 270. The attachment member 250 is not limited to a particular number of attachment holes 270. In certain embodiments, the attachment member 250 has therein two attachment holes 270. The attachment member 250 is not limited to a particular positioning of the attachment holes 270. In certain embodiments, the attachment holes are positioned along the outer edge of the attachment member 250. In certain embodiments, the attachment holes 270 are configured to receive threaded fasteners (e.g., screws such as bone screws). In certain embodiments, as a cannula device 100 is positioned within a bone hole (e.g., burr hole), threaded fasteners are inserted into the attachment holes 270 such that the cannula device 100 is secured into a fixed position within the bone hole. Also in FIG. 4A, shown in dashed lines, is the proximal end 130 of the tubular extension that would be underneath the attachment member 250. The area of the attachment member that extends beyond the proximal end 130 is referred to as the overhang portion 255 of the attachment member. As shown in FIG. 4A, the attachment holes 270 are located in this overhang portion 255 of attachment member 250.

FIG. 4B shows a top-down perspective of an attachment member 250 having attachment member openings 260, an overhang portion 255, and attachment holes 270. In this attachment member 250 embodiment, the size of the overhang portion 255 is limited to the area surrounding the attachment holes 270. As such, the overhang portion 255 is not limited to a particular amount of encompassment above the proximal end 130 of the tubular extension. In certain embodiments, as shown in FIG. 4A, the overhang portion 255 extends beyond the entire proximal end 130 of the tubular extension. In other embodiments, as shown in FIG. 4B, the overhang portion 255 only extends beyond the portion of the proximal end 130 near the attachment holes 270.

FIGS. 4C and 4D show a top-down perspective of an attachment member 250 having an overhang portion 255 and attachment holes 270 positioned above the tubular extension proximal end 130. As shown in FIG. 4C, the shape of the attachment member 250 assumes a ring configuration such that the tubular extension proximal end 130 is exposed. As shown in 4D, the shape of the attachment member 250 is tabular such that the overhang portion 255 only encompasses the area surrounding the attachment holes 270.

Referring again to FIG. 1, the attachment member 250 is not limited to a particular manner of connection with the tubular extension body proximal end 130. In some certain embodiments, the attachment member 250 is rigidly connected to the tubular extension proximal end 130 at a predetermined angle (e.g., 0 to 180 degrees) in relation to the tubular extension proximal end 130. In such embodiments, as the cannula device 100 is positioned within a bone hole (e.g., a burr hole) the angle of the attachment member 250 in relation to the tubular extension 110 remains fixed. In other certain embodiments, the attachment member 250 is connected to the tubular extension proximal end 130 in a flexible manner (e.g., via a movable hinge or other flexible component) such that the attachment member is able to assume any desired angle in relation to the tubular extension proximal end 130 (e.g., 0 to 250 degrees). In such embodiments, as a cannula 100 is positioned within a bone hole (e.g., burr hole) the angle of the attachment member 250 in relation to the tubular extension 110 may be adjusted to a desired angle (e.g., upon securing of the attachment member 250 with the bone hole, the tubular extension 110 may be adjusted to any desired angle).

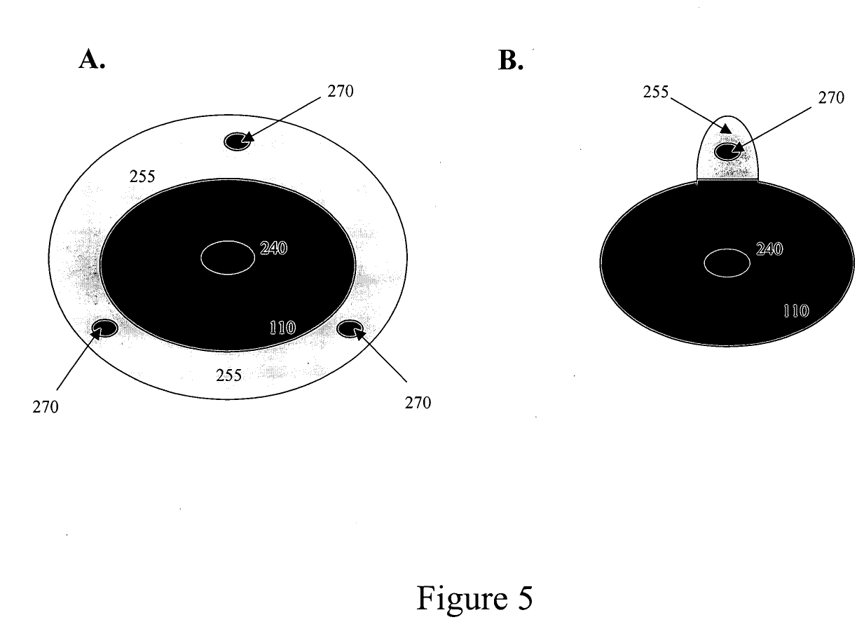

FIGS. 5A and 5B show a bottom-up perspective of a cannula device 100 having a tubular extension 110, a tubular extension hollow tract exit 240, and an attachment member with the overhang portion 255 and attachment holes 270. As shown, the overhang portion 255 extends beyond the tubular extension 110.

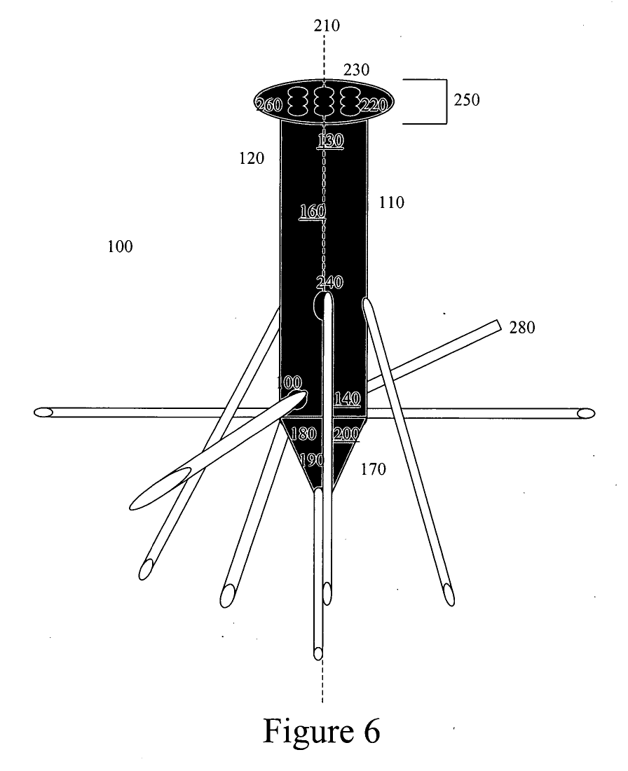

FIG. 6 shows a side perspective of a cannula device 100 having an attachment member 250 with attachment member openings 260; a tubular extension 110 with a tubular extension body 120 having a tubular extension body proximal end 130, a tubular extension body distal end 140, and a tubular extension body exterior surface 160; a tubular extension nose 170 having a tubular extension nose proximal end 180, a tubular extension nose distal end 190, and a tubular extension nose exterior surface 200; a tubular extension longitudinal axis 210; and multiple tubular extension hollow tracts 220 having tubular extension hollow tract entrances 230 and tubular extension hollow tract exits 240. As shown, the cannula device 100 has surgical catheters 280 positioned within tubular extension hollow tracts 220. The present invention is not limited to the use of a particular type of surgical catheter. In certain embodiments, the surgical catheters useful with the multiple-tract cannulas 100 include flexible catheters capable of delivering pharmaceutical agents (e.g., drugs). The tubular extension hollow tract exits 240 may assume varied angles in relation to the tubular extension longitudinal axis 210 thereby allowing the surgical catheters 280 to project at a multitude of angles for optimal delivery of, for example, pharmaceutical agents.

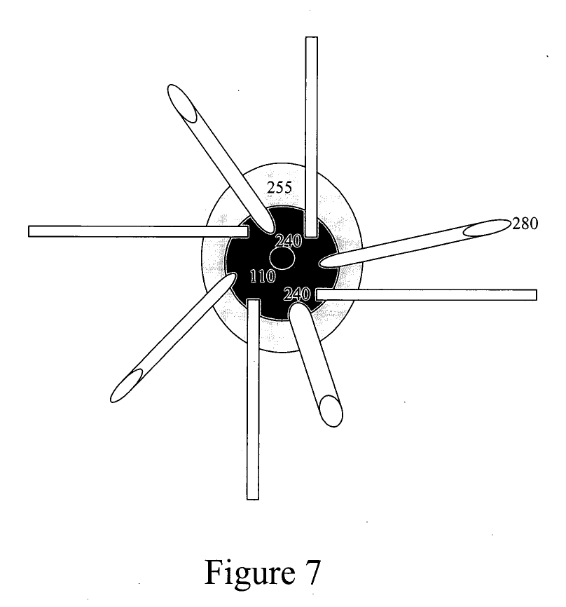

FIG. 7 shows a bottom-up perspective of a cannula device 100 having a tubular extension 110, tubular extension hollow tract exits 240, and an attachment member 250 with an overhang portion 255. As shown, the cannula device 100 has surgical catheters 280 exiting the tubular extension hollow tract exits 240 at a multitude of angles.

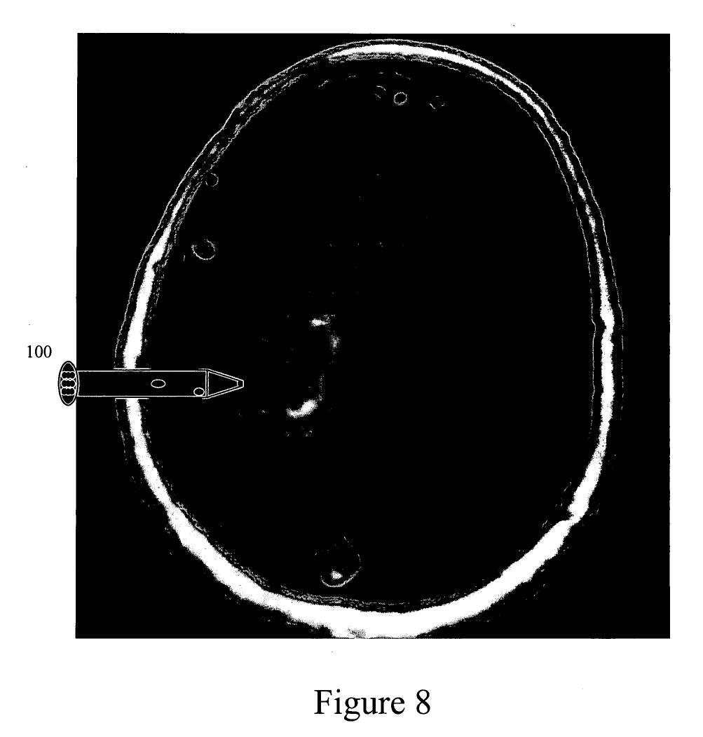

FIG. 8 shows a cannula device 100 positioned in the proximity of a brain mass. As shown, the cannula device 100 is configured for positioning in the vicinity of a desired body cavity for optimal delivery of pharmaceutical agents.

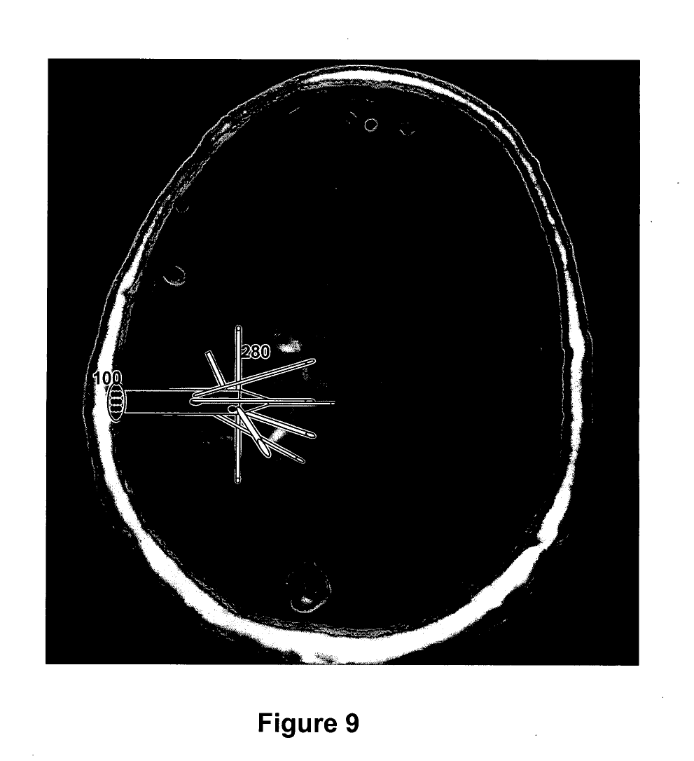

FIG. 9 shows a surgical catheters 280 positioned within a cannula device 100 in the proximity of a brain mass. As shown, the tubular extension hollow tract exits are shown assuming varied angles in relation to the tubular extension longitudinal axis thereby allowing the surgical catheters 280 to project at a multitude of angles.

The devices of the present invention may be combined within various kit embodiments. For example, the present invention provides kits comprising the cannula device along with any one or more accessory agents (e.g., surgical catheter, screws, glue, or other fastening agents). The present invention is not limited to any particular accessory agent. For example, accessory agents include, but are not limited to, surgical catheters, fastening agents, and a convection-enhanced drug delivery apparatus. Additionally, the present invention contemplates kits comprising instructions (e.g., surgical instructions, pharmaceutical instructions) along with the cannula devices of the present invention and/or a pharmaceutical agent (e.g., a neurological medication).

The devices of the present invention may be used in any surgical or neurosurgical technique (e.g., surgical method). In certain embodiments, a cannula device of the present invention is used to deliver pharmaceutical agents at a variety of angles to a region of the brain or other tissue type. As such, the devices of the present invention may be used, for example, to treat any kind of brain abnormality (e.g., brain tumor), or other type of surgically addressable condition. Any type of brain tumor may be treated with the devices of the present invention, including but not limited to, astrocytoma tumors, glioma tumors, atypical teratoid/rhabdoid tumors, brain stem gliomas, choroid plexus tumors, craniopharyngiomas, ependymoma tumors, ganglioglioma tumors, germ cell tumors, gliomatosis cerbri tumors, infant brain tumors, medulloblastoma tumors, oligodendroglioma tumors, and optic pathway tumors. In other embodiments, the devices of the present invention are used to deliver pharmaceutical agents to any location within a body (e.g., the liver).

### EXAMPLE

This example describes a contemplated surgical method for delivering pharmaceutical agents (e.g., drugs) to a brain tumor utilizing a cannula device of the present invention. While this example describes the delivery of pharmaceutical agents to a brain tumor, the technique may be applied to any unwanted brain mass or unwanted brain lesion. First, a burr hole is placed within the cranium. Second, a cannula device of the present invention is secured within the burr hole. The cannula device provides multiple points of access for the insertion of surgical catheters. Third, surgical catheters are passed through the cannula device in the vicinity of the brain tumor at a variety of angles. Fourth, pharmaceutical agents are delivered to the brain tumor through the surgical catheters.

All publications and patents mentioned in the above specification are herein incorporated by reference. Although the invention has been described in connection with specific certain embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A cannula device comprising a tubular extension and an attachment member,
wherein said tubular extension comprises proximal and distal ends and an exterior surface, said tubular extension having a longitudinal axis and having therein at least two hollow tracts running from said proximal end and through said exterior surface, wherein said tubular extension is inflexible, and
wherein said attachment member is connected to said proximal end of said tubular extension, wherein said attachment member comprises an overhang portion that extends beyond said exterior surface of said tubular extension, and wherein said overhang portion has therein a plurality of holes configured to receive fastening agents.

2. The cannula device of claim 1, wherein the diameter of said hollow tracts is less than 5 mm.

3. The cannula device of claim 1, wherein the diameter of said tubular extension is less than 5 cm in diameter.

4. The cannula device of claim 1, wherein said tubular extension has two of said hollow tracts.

5. The cannula device of claim 1, wherein the number of said hollow tracts within said tubular extension is selected from the group consisting of: 3, 4, 5, 6, 7, 8, 9, and 10.

6. The cannula device of claim 1, wherein at least two of said hollow tracts extend through said tubular extension in a non-parallel manner with respect to one another.

7. The cannula device of claim 1, wherein at least two of said hollow tracts extend through said tubular extension in separate spiral trajectory paths.

8. The cannula device of claim 1, wherein said tubular extension is less than 7.5 centimeters in length.

9. The cannula device of claim 1, wherein said tubular extension is between 1 and 5 cm in length.

10. The cannula device of claim 1, wherein said attachment member is rigidly connected to said proximal end of said tubular extension.

11. The cannula device of claim 10, wherein said attachment member is connected at an angle between 0 and 180 degrees in relation to said longitudinal axis.

12. The cannula device of claim 1, wherein said attachment member is flexibly attached to said proximal end of said tubular extension.

13. The cannula device of claim 12, wherein said attachment member may be adjusted to an angle between 0 and 180 degrees in relation to said longitudinal axis.

14. The cannula device of claim 13, further comprising a hinge, wherein said attachment member attaches to said proximal end of said tubular extension with said hinge.

15. The cannula device of claim 1, wherein said hollow tracts are configured to receive surgical catheters.

16. A cannula device comprising a tubular extension and an attachment member, wherein said tubular extension comprises proximal and distal ends and an exterior surface, said tubular extension having a longitudinal axis and having therein at least two hollow tracts running from said proximal end and through said exterior surface, wherein said hollow tracks extend through said tubular extension in a non-parallel manner with respect to one another, and
wherein said attachment member is connected to said proximal end of said tubular extension, wherein said attachment member comprises an overhang portion that extends beyond said exterior surface of said tubular extension, and wherein said overhang portion has therein a plurality of holes configured to receive fastening agents.

17. The cannula device of claim 16, wherein the diameter of said hollow tracts is less than 5 mm.

18. The cannula device of claim 16, wherein the diameter of said tubular extension is less than 5 cm in diameter.

19. The cannula device of claim 16, wherein said tubular extension has two of said hollow tracts.

20. The cannula device of claim 16, wherein the number of said hollow tracts within said tubular extension is selected from the group consisting of: 3, 4, 5, 6, 7, 8, 9, and 10.

21. The cannula device of claim 16, wherein said tubular extension is less than 7.5 cm in length.

22. The cannula device of claim 16, wherein the diameter of said tubular extension is between one and five centimeters in diameter.

23. The cannula device of claim 16, wherein said tubular extension is inflexible.

24. The cannula device of claim 16, wherein said attachment member is rigidly connected to said proximal end of said tubular extension.

25. The cannula device of claim 24, wherein said attachment member is connected at an angle between 0 and 180 degrees in relation to said longitudinal axis.

26. The cannula device of claim 16, wherein said attachment member is flexibly attached to said proximal end of said tubular extension.

27. The cannula device of claim 16, further comprising a hinge, wherein said attachment member attaches to said proximal end of said tubular extension with said hinge.

28. The cannula device of claim 16, wherein said hollow tracts are configured to receive surgical catheters.
